(19) United States
(12) Patent Application Publication
(10) Pub. No.: US 2015/0290188 A1
(43) Pub. Date: Oct. 15, 2015

(54) PHARMACEUTICAL COMPOSITIONS CONTAINING ENANTIOMERICALLY PURE AND/OR RACEMIC MIXTURES OF CHIRAL PIPERAZINE COMPOUNDS AND METHODS OF TERMINATING ACUTE EPISODES OF CARDIAC ARRHYTHMIA, RESTORING NORMAL SINUS RHYTHM, PREVENTING RECURRENCE OF CARDIAC ARRHYTHMIA AND MAINTAINING NORMAL SINUS RHYTHM IN MAMMALS THROUGH ADMINISTRATION OF SAID COMPOSITIONS

(71) Applicant: ChanRx Corporation, Cleveland, OH (US)

(72) Inventor: Arthur M. Brown, Brecksville, OH (US)

(21) Appl. No.: 14/437,493

(22) PCT Filed: Oct. 11, 2013
(86) PCT No.: PCT/US2013/064684

§ 371 Date: Apr. 21, 2015

Related U.S. Application Data
(60) Provisional application No. 61/818,165, filed on May 1, 2013.
(60) Provisional application No. 61/712,620, filed on Oct. 11, 2012.
(60) Provisional application No. 61/843,484, filed on Jul. 8, 2013.

(51) Int. Cl. A61K 31/495

(52) U.S. Cl. CPC A61K 31/495

(57) ABSTRACT

Disclosed embodiments are related to compositions of a chiral piperazine compounds including compositions comprising enantiomerically pure or racemic mixtures of said piperazine compounds, and one or more diluents, disintegrants, binders and lubricants, and the processes for their preparation thereof.

### FIELD OF THE INVENTION

Presently disclosed embodiments are related to pharmaceutical compositions comprising enantiomerically pure and/or racemic mixtures of chiral piperazine compounds and processes for the preparation thereof. Presently disclosed embodiments particularly relate to pharmaceutical compositions that include piperazine compounds in combination with one or more diluents, disintegrants, binders and/or lubricants.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732.) Vanoxerine has also been used for treating and preventing cardiac arrhythmia in mammals. (See U.S. Pat. No. 6,743,797 and U.S. Pat. No. 7,700,600.)

It is desirable to identify piperazine compounds and compositions containing the same, their enantiomers, and compositions comprising racemic mixtures and enantiomerically pure compositions of said piperazine compounds for use in treatment of cardiac arrhythmias.

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp. 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, they have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A third class of anti-arrhythmic action: effects on atrial and ventricular intracellular potentials and other pharmacological actions on cardiac muscle of MJ 1999 and AH 3747, ” Br. J. Pharmacol 39:675-689 (1970), and Singh B. N., Vaughan Williams E. M., “The effect of amiodarone, a new anti-anginal drug, on cardiac muscle,” Br. J. Pharmacol 39:657-667 (1970)), but these are not selective Class III agents.

Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)) Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (T_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr}, (rapidly activating and deactivating) and I_{Ks} (slowly activating and deactivating). (Sanguinetti and Jurkiewicz, “Two components of cardiac delayed rectifier K+ current. Differential sensitivity to block by Class III anti-arrhythmic agents,” J Gen Physiol 96:195-215 (1990)). I_{Kr}, is also the product of the human ether-a-go-go gene (hERG). Expression of hERG cDNA in cell lines leads to production of the hERG current which is almost identical to I_{Kr} (Curran et al., “A molecular basis for cardiac arrhythmia: hERG mutations cause long QT syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6yl], (+)-, monochloride (MK-499) predominantly, if not exclusively, block I_{Kr}. Although, amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, is as a nonspecific adrenergic blocker, acts as an inhibitor of the enzyme phospholipase, and causes pulmonary fibrosis (Nademanee, K. “The Amiodarone Odessey”. J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness by prolonging APD, prevents and/or terminates reentrant arrhythmias. Most selective, Class III antiarrhythmic agents currently in development, such as d-sotalol and dofetilide predominantly, if not exclusively, block I_{Kr}, the rapidly activating component of I_{K} found both in atrium and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M. “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supra-ventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced effectiveness and dangers of reverse use dependence,” Circulation, 81:686-690 (1990); Sadanaga et al., “Clinical evaluation of the use-dependent QRS prolongation and the reverse use-dependent QT prolongation of class III anti-arrhythmic agents and their value in predicting efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-dependent prolongation of cardiac action potentials by a methanesulfonanilide class III anti-arrhythmic agent: Specific block of rapidly activating delayed rectifier K+ current by dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

In view of the problems associated with current anti-arrhythmic agents, there remains a need for an effective treatment of cardiac arrhythmias in mammals with a piperazine compound including enantiomerically pure and racemic mixtures of chiral piperazine compounds. The newly discovered formulations preferably use a minimal number of excipients and use pharmaceutical grade excipients that are inexpensive, readily available, and that facilitate cost-effective manufacture on a commercial scale for the treatment of among other

### SUMMARY

Accordingly, it is an object of the present invention to provide compounds and pharmaceutical compositions for preventing or treating cardiac arrhythmia in mammals, particularly humans.

In accordance with these and other objects, a first embodiment of the present invention comprises piperazine compounds having the structure:

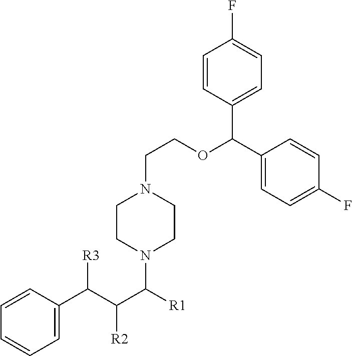

wherein each of R1, R2, and R3 is independently a hydrogen atom or a hydroxyl group, provided that not all of R1, R2 and R3 are the same, and further provided that R1 and R2 are not both a hydroxyl group and that R2 and R3 are not both a hydroxyl group, and wherein said hydroxyl group at either the R1, R2, or R3 position renders the compound chiral.

Embodiments of the present disclosure relate to chiral piperazine compounds and compositions comprising enantiomerically pure and racemic mixtures of the piperazine compounds. In particular, piperazine compounds are admixed with various excipients to formulate a solid dose of vanoxerine. In certain embodiments, the solid dose is in tablet form; in other embodiments, it is in capsule form.

An additional aspect of the present disclosure includes processes for the preparation of novel piperazine formulations. In particular, the processes involve preparation of a solid dosage form of a piperazine compound comprising either an enantiomerically pure or a racemic mixture of a piperazine compound, preferably by wet mixing the piperazine compound and excipients with water, followed by drying and milling of the granulated mixture.

Other aspects of the present disclosure include methods of treatment of cardiac arrhythmias comprising administering a therapeutically effective amount of an enantiomerically pure or a racemic mixture of a piperazine compound.

Other aspects of the present invention comprises methods for terminating acute episodes of cardiac arrhythmia, such as atrial fibrillation or ventricular fibrillation, in a mammal, such as a human, by administering to that mammal at least an effective amount of an enantiomerically pure or a racemic mixture of a piperazine compound to terminate an acute episode of cardiac arrhythmia.

Other aspects of the present invention is directed to a method for restoring normal sinus rhythm in a mammal, such as a human, exhibiting cardiac arrhythmia by administering at least an effective amount of an enantiomerically pure or a racemic mixture of a piperazine compound to restore normal sinus rhythm.

Other aspects of the present invention is directed to a method for maintaining normal sinus rhythm in a mammal, such as a human, by administering at least an effective amount of an enantiomerically pure or a racemic mixture of a piperazine compound to maintain normal sinus rhythm in a mammal that has experienced at least one episode of cardiac arrhythmia.

Other aspects of the present invention is directed to a method for preventing a recurrence of an episode of cardiac arrhythmia in a mammal, such as a human, by administering to that mammal at least an effective amount of an enantiomerically pure or a racemic mixture of a piperazine compound to prevent a recurrence of cardiac arrhythmia.

### BRIEF DESCRIPTION OF THE DRAWINGS

FIGS. 1a and 1b are drawings of one embodiment of the invention described herein.

FIGS. 2a, and 2b are drawings of an embodiment of the invention described herein.

FIGS. 3a and 3b are drawings of an embodiment of the invention described herein.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein “a piperazine compound” and variations thereof refers to compounds having the structure:

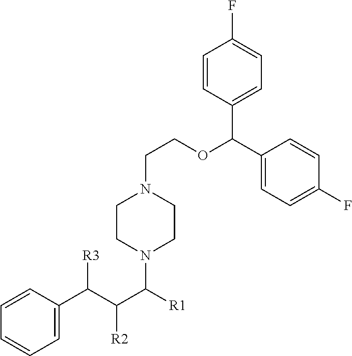

wherein each of R1, R2, and R3 is independently a hydrogen atom or a hydroxyl group, provided that not all of R1, R2 and R3 are the same, and further provided that R1 and R2 are not both a hydroxyl group and that R2 and R3 are not both a hydroxyl group. and wherein said hydroxyl group at either the R1, R2, or R3 position renders the compound chiral.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein—described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either a piperazine compound or a pharmaceutically acceptable composition comprising a piperazine compound.

As used herein, “racemic mixture” means about a 50:50 mixture of R/S enantiomers.

As used here, “enantiomerically pure” means a composition comprising about 99.9% of a single enantiomer and about less than 0.01% of the corresponding enantiomer, so that a composition comprises about 99.9:0.01 R/S or 99:1 S/R, where the goal is to have a composition comprising a substance comprising 100% of one enantiomer and 0% of the other enantiomer. However circumstances may permit about 99% and about 1% for a 99:1 R/S, S/R ratio.

The chiral piperazine compounds can be synthesized by one of ordinary skill in the art using starting products and reactants known to one of ordinary skill in the art.

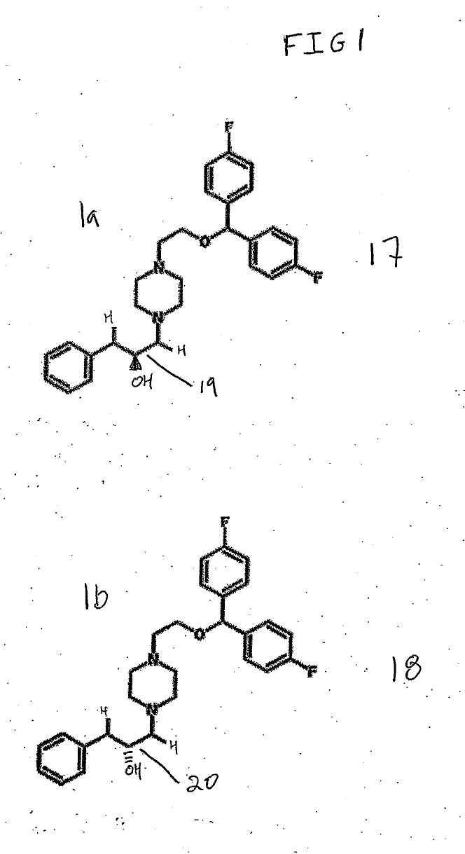

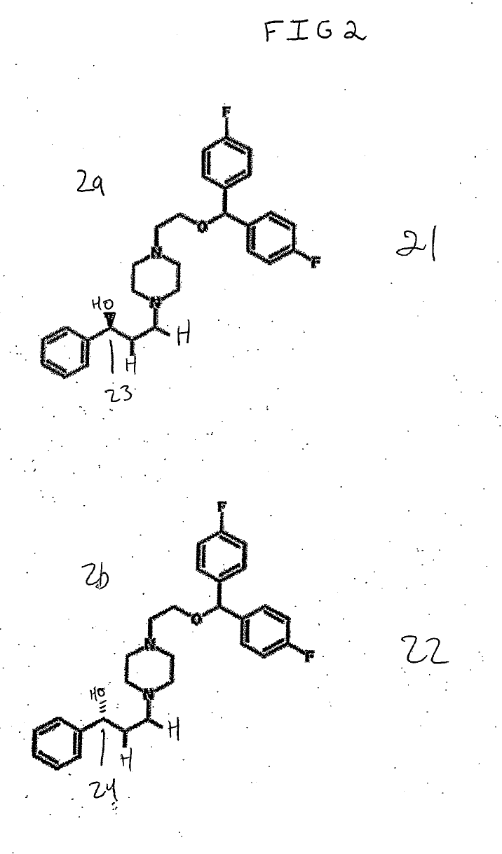

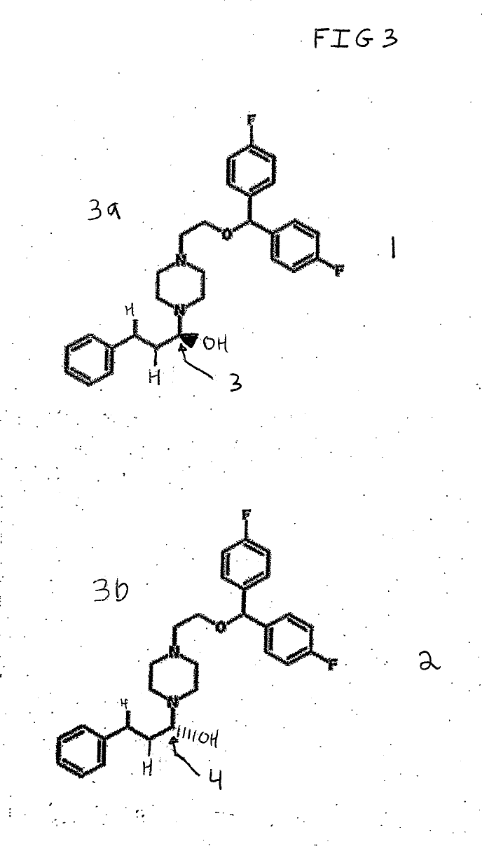

In view of the figures, FIGS. 1, 2, and 3 identify chiral piperazine compounds. FIG. 1a shows a piperazine compound 17 having a chiral carbon 19. FIG. 1b shows a piperazine compound 18 having a chiral carbon 20. FIG. 2a shows a piperazine compound 21 having a chiral carbon 23. FIG. 2b shows a piperazine compound 22 having a chiral carbon 24. FIG. 3a shows a piperazine compound 1 having chiral carbon 3. FIG. 3b shows piperazine compound 2 having a chiral carbon 4.

Preferred embodiments include pharmaceutical compositions of an enantiomerically pure or a racemic mixture of a piperazine compound, with one or more excipients, such as those pharmaceutically acceptable diluents, disintegrants, binders and lubricants known and available to those skilled in the art. Preferably, the excipients meet the standards of the National Formulary (“NF”) and/or United States Pharmacopoeia (“USP”). In a particular preferred embodiment, there is provided a pharmaceutical composition comprising a piperazine compound with one or more diluents, disintegrants, binders and/or lubricants.

The excipients are selected to ensure the delivery of a consistent amount of an enantiomerically pure or a racemic mixture of a piperazine compound, in a convenient unit dosage form and to optimize the cost, ease and reliability of the manufacturing process. All excipients must be inert, organoleptically acceptable, and compatible with an enantiomerically pure or a racemic mixture of a piperazine compound. The excipients used in a solid oral formulation commonly include fillers or diluents, binders, disintegrants, lubricants, antiadherents, glidants, wetting and surface active agents, colors and pigments, flavoring agents, sweeteners, adsorbents, and taste-maskers.

Diluents are typically added to a small amount of the active drug to increase the size of the tablet. A suitable diluent for use in the inventive compositions is lactose, which exists in two isomeric forms, alpha-lactose or beta-lactose, and can be either crystalline or amorphous. Various types of lactose include spray dried lactose monohydrate (such as Super-Tab™), alpha-lactose monohydrate (such as Fast Flo®), anhydrous alpha-lactose, anhydrous beta-lactose, and agglomerated lactose. Other diluents include sugars, such as compressible sugar NF, dextrose excipient NF, and dextrates NF. A preferred diluent is lactose monohydrate (such as Fast Flo®). Other preferred diluents include microcrystalline cellulose (such as Avicel® PH, and Ceolus™) and microfine cellulose (such as Elcema®).

Suitable diluents also include starch and starch derivatives. Starches include native starches obtained from wheat, corn, rice and potatoes. Other starches include pregelatinized starch NF, and sodium starch glycolate NF. Starches and starch derivatives can also function as disintegrants. Other diluents include inorganic salts, including, but not limited to, dibasic calcium phosphate USP (such as Di-Tab® and Emcompress®), tribasic calcium phosphate NF (such as Tri-Tab® and Tri-Cafos®), and calcium sulfate NF (such as Compactrol®). Polyols such as mannitol, sorbitol, and xylitol may also serve as diluents. Many diluents can also function both as disintegrants and as binders, and these additional properties should be taken into account when developing particular formulations.

Disintegrants may be included to break larger particles, such as tablets, granules, beads, nonpareils and/or dragees, into smaller particles comprising the active pharmaceutical ingredient and, optionally, other excipients which may facilitate dissolution of the active ingredient and/or enhance bioavailability of the active ingredient. Starch and starch derivatives, including cross-linked sodium salt of a carboxymethyl ether of starch (such as sodium starch glycolate NF, Explotab®, and Primogel®) are useful disintegrants. A preferred disintegrant is cross-linked sodium carboxymethyl cellulose (such as Croscarmellose Sodium NF, Ac-Di-Sol®). Other suitable disintegrants include, but are not limited to, cross-linked polyvinylpyrrolidone (such as Crospovidone NF) and microcrystalline cellulose (such as Avicel® PH).

Binders may also be used as an excipient, particularly during wet granulation processes, to agglomerate the active pharmaceutical ingredient and the other excipients. In all formulation, whether prepared by wet or dry granulation, a particular binder is generally selected to improve powder flow and/or to improve compactibility. Suitable binders include, but are not limited to, cellulose derivatives, such as microcrystalline cellulose NF, methylcellulose USP, carboxymethycellulose sodium USP, hydroxypropyl methylcellulose USP, hydroxyethyl cellulose NF, and hydroxypropyl cellulose NF. Other suitable binders include polyvidone, polyvinyl pyrrolidone, gelatin NF, natural gums (such as acacia, tragacanth, guar, and pectin), starch paste, pregelatinized starch NF, sucrose NF, corn syrup, polyethylene glycols, sodium alginate, ammonium calcium alginate, magnesium aluminum silicate and polyethylene glycols.

Lubricants may be used, particularly in tablet formulations, to prevent sticking of the ingredients and/or dosage form to the punch faces and to reduce friction during the compression stages. Suitable lubricants include, but are not limited to, vegetable oils (such as corn oil), mineral oils, polyethylene glycols (such as PEG-4000 and PEG-6000), salts of stearic acid (such as calcium stearate and sodium stearyl fumarate), mineral salts (such as talc), inorganic salts (such as sodium chloride), organic salts (such as sodium benzoate, sodium acetate, and sodium oleate) and polyvinyl alcohols. A preferred lubricant is magnesium stearate.

In preferred embodiments, an enantiomerically pure or a racemic mixture of a piperazine compound generally comprises from about 20-50% by weight of the pharmaceutical composition, more preferably from about 25-40% and most preferably from about 30-35%. In a racemic mixture, the R/S concentrations are about 50:50, but it is suitable for compositions to be about 60:40 or even about 70:30 or about 80:20 in some circumstances. In embodiments where an enantiomerically pure composition is utilized, about 100:0 is preferred, but about 99:1 or about 98:2 or about 97:3, or about 95:5, or about 90:10 are acceptable in certain embodiments.

Preferably, the inventive composition also comprises a diluent which is lactose monohydrate, a binder which is microcrystalline cellulose; a disintegrant which is a cross-linked sodium carboxymethyl cellulose; a flowing agent which is colloidal silicon dioxide, and a lubricant which is magnesium stearate. Suitable amounts of each excipient may be determined empirically by one skilled in the art considering such factors as the particular mode of administration (e.g. oral, sublingual, buccal, etc.), amount of active ingredient (e.g. 50 mg, 60 mg, 80 mg, 100 mg, 150 mg, etc.), particular patient (e.g. adult human, human child, etc.) and dosing regimen (e.g. once a day, twice a day, etc.).

In certain preferred embodiments, the inventive compositions may contain lactose monohydrate (e.g. Fast Flo® #316) from about 30-60% of the composition by weight, more preferably from about 35-50% and most preferably from about 40-45%.

In certain preferred embodiments, the inventive compositions may contain microcrystalline cellulose (e.g. Avicel® PH 102)from about 5-30% by weight of the composition, more preferably from about 10-25% and most preferably from about 15-20% by weight,

In certain preferred embodiments, the inventive compositions may contain cross-linked sodium carboxymethyl cellulose (e.g. Ac-Di-Sol®) from about 0.1-10% by weight of the composition, more preferably from about 0.5-5% and most preferably from about 1-3% by weight,

In certain preferred embodiments, the inventive compositions may contain colloidal silicon dioxide (e.g. Aerosil® A-200) from about 0.02 to about 1% by weight of the composition, more preferably form about 0.1 to about 0.6% and most preferably from about 0.2-0.4% by weight.

In certain preferred embodiments, the inventive compositions may contain magnesium stearate from about 0.02 to about 1% by weight of the composition, more preferably form about 0.1 to about 0.6% and most preferably from about 0.2-0.4% by weight.

Solid dosage forms of an enantiomerically pure or a racemic mixture of a piperazine compound can be prepared using any of the methods and techniques known and available to those skilled in the art.

For example, a solid dosage form of an enantiomerically pure or a racemic mixture of a piperazine compound can be prepared by wet mixing a piperazine compound and excipients with water, drying and milling the granulated mixture. In certain embodiments, the final mixture is compressed into a tablet. In other embodiments, the final mixture is encapsulated.

In particular, the process comprises the steps of: (a) dry blending of an enantiomerically pure or a racemic mixture of a piperazine compound, and one or more excipients to form a dry mixture; (b) wetting the dry mixture with water, preferably with purified water, to form a wet granulation mixture; (c) drying the wet granulation mixture to form a dried granulation mixture; (d) milling the dried granulation mixture to form a milled granulation mixture; (e) mixing a lubricant in the milled granulation mixture to give a final blended mixture; (f) preparing the final blended mixture in a solid dosage form suitable for oral administration.

In certain preferred embodiments, the final blended mixture is compressed into tablets. In other preferred embodiments, the final blended mixture is enclosed in a capsule.

Specifically, in step (a), an enantiomerically pure or a racemic mixture of a piperazine compound is blended with all excipients in the final formulation, other than the lubricant. In particular, an enantiomerically pure or a racemic mixture of a piperazine compound is thoroughly dry blended with the diluent(s), disintegrant(s) and binder to form a uniform dry mixture. Blenders appropriate for large scale dry blending include twin shell blenders, double cone blenders, and ribbon blenders. Ribbon blenders have the advantage of being used in continuous-production procedures. High-speed, high shear mixers may also be used and offer the advantage of shorter mixing times. The dry mixture may also be granulated, milled into a fine powder, passed through a mesh screen, or micronized, if necessary. Preferably, the dry blending was performed in high shear granulators.

The resulting dry mixture is then wetted with a wetting agent to form a wet granulation mixture in step (b). The wetting agent is typically added over time, usually from about 1 to about 15 minutes, with continuous mixing. Typically, the wetting agent is added to the blender used in the dry blending step. Preferably the wet granulation is carried out in a high shear granulator. In certain embodiments, the wetting agent is an aqueous-based solution. Preferably, the wetting agent is water without any additional solvents, and in particular, without organic solvents. More preferably, the water is purified water.

The type and amount of wetting agent, rate of addition of wetting agent, and the mixing time influences the structure of the granules. The different types of granules, such as pendular, funicular, capillary, etc., can be manipulated to achieve the desired density, porosity, texture and dissolution pattern of the granules, which in turn, determines the compressibility, hardness, disintegration, and consolidation characteristics of the dried mixture.

The wet granulation mixture is then dried in step (c) to form a dried granulation mixture with an appropriate moisture content. In certain embodiments, the drying means include a fluid bed or tray dryers. Fluid bed drying yield shorter drying times, in the range from 1 to 3 hours, while tray drying averages 10 to 13 hours. Preferably, the wet granulation mixture is dried in a fluid bed, for preferably about 1-3 hours. Fluid bed drying has the added advantages of better temperature control and decreased costs. The method of drying, drying time, and moisture content are critical to avoid decomposition, chemical migration, and other adverse physical characteristics of dried mixture which can affect the dosage form performance.

The dried granulation mixture is subsequently milled in step (d) to form a milled granulation mixture. The particle size of the dried granulation mixture is reduced to achieve an appropriate particle size distribution for the subsequent processes. In certain embodiments, milling is achieved using a high shear impact mill (such as Fitzpatrick) or a low shear screening mill (such as Comil). The dried granulation mixture may also be screened to select the desired granule size.

In the next step (e), the lubricant was blended with the dried granulation mixture to give a final blended mixture. In certain embodiments, a V blender or bin blenders are used. A preferred blender is a V-shell PK blender. A gentle blending is preferred, such that each granule covered with the lubricant, while minimizing the breaking up of the granules. Increased breaking of the granules results in fine powder, or “fines”. A high fine content results in variations of weight and density during compression into a tablet, as well as increases the need for cleaning of the compression machinery.

The final blended mixture is then prepared in a solid dosage form suitable for oral administration. Solid dosage forms include tablets, capsules, pills, troches, cachets, and the like. In one embodiment, the final blended mixture is compressed into a tablet. The compression machinery typically contains two steel punches within a steel die cavity. The tablet is formed when pressure is exerted on the dried granulation mixture by the punches in the cavity, or cell.

Tableting machines include single-punch machines, rotary tablet machines, gravity feed, and powder assisted machines. Preferably, gravity feed or powder assisted machines are used. Rotary machines operating at high speeds suitable for large-scale production include double rotary machines and single rotary machines. Tablets can also include sugar-coated tablets, film-coated tablets, enteric-coated tablets, multiple-compressed tablets, controlled-release tablets, tablets for solution, effervescent tablets or buccal and sublingual tablets.

Compressed tablets may be characterized by a number of specifications, including diameter size, shape, thickness, weight, hardness, friability, disintegration time, and dissolution characteristics. The tablets preferably have weights, friability and dissolution rates in accordance with USP standards.

In other embodiments, the final blended mixture is enclosed in capsules, preferably hard gelatin capsules. The hard gelatin capsules are commercially available, and are generally made from gelatin, colorants, optionally an opacifying agent such as titanium dioxide, and typically contain 12-16% water. The hard capsules can be prepared by filling the longer end of the capsule with the final blended mixture, and slipping a cap over the top using mG2, Zanasi, or Hofliger and Karg (H&K) machines.

In an alternative embodiment, the present invention provides for a process of preparing a solid dose form of an enantiomerically pure or a racemic mixture of a piperazine compound by dry mixing an enantiomerically pure or a racemic mixture of a piperazine compound with the excipients. In certain embodiments, the mixture is compressed into a tablet. In other embodiments, the mixture is encapsulated.

In particular, the process comprises the steps of: (a) dry blending of an enantiomerically pure or a racemic mixture of a piperazine compound and one or more excipients to form a dry mixture; (b) mixing a lubricant in the dry mixture to give a final blended mixture; (c) preparing the final blended mixture in a solid dosage form suitable for oral administration.

In certain preferred embodiments, the final blended mixture is compressed into tablets. In other preferred embodiments, the final blended mixture is enclosed in a capsule.

Specifically, in step (a), an enantiomerically pure or a racemic mixture of a piperazine compound is blended with all excipients in the final formulation, other than the lubricant. Preferably, an enantiomerically pure or a racemic mixture of a piperazine compound is thoroughly dry blended with the diluent(s), disintegrant(s) and a binder to form a uniform dry mixture. Blenders appropriate for large scale dry blending include twin shell blenders, double cone blenders, V blenders or bin blenders. A preferred blender is a V-shell PK blender. High-speed, high shear mixers may also be used. The dry mixture may also be granulated, milled into a fine powder, passed through a mesh screen, or micronized, if necessary.

In the next step (b), the lubricant was blended with the dry mixture to give a final blended mixture. In certain embodiments, a V blender or bin blenders are used. A preferred blender is a V-shell PK blender.

The final blended mixture is then prepared in a solid dosage form suitable for oral administration. Solid dosage forms include tablets, capsules, pills, troches, cachets, and the like. In one embodiment, the final blended mixture is compressed into a tablet. In another embodiment, the final blended mixture is enclosed in capsules, preferably hard gelatin capsules.

Other aspects of the invention also include use of these compositions for the treatment of a disease or disorder in a subject in need thereof comprising administering to the subject a therapeutically effective amount of the compositions of the present invention. In particular, the present compositions are useful in the treatment of cocaine addiction, acute effects of cocaine, cocaine cravings, Parkinsonism, acromegaly, hyperprolactinemia and diseases arising from a hypofunction of the dopaminergic system, and cardiac arrhythmia.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

Formulation of a 100 mg enantiomerically pure piperazine compound comprising the S enantiomer, Capsule

| | Amount per | |
|---|---|---|
| Components | tablet (mg) | Amount per batch (mg) |
| The S enantiomer of a | 100.0 | 120.0 |
| piperazine compound | | |
| Lactose Monohydrate, NF | 121.00 | 145.20 |
| Microcrystalline Cellulose, NF | 51.00 | 61.20 |
| Croscarmellose Sodium, NF | 6.00 | 7.20 |
| Colloidal Silicon Dioxide, NF | 1.00 | 1.20 |
| Magnesium Stearate, NF | 1.00 | 1.20 |
| Total Tablet Weight | 300.0 | 336.0 |

### Example 2

Formulation of a 200 mg Capsule of an enantiomerically pure composition comprising the R enantiomer of a piperazine compound:

| | Amount per | |
|---|---|---|
| Components | tablet (mg) | Amount per batch (mg) |
| The R enantiomer of a | 200.0 | 240.0 |
| piperazine compound | | |
| Lactose Monohydrate, NF | 242.00 | 290.40 |
| Microcrystalline Cellulose, NF | 102.00 | 122.40 |
| Croscarmellose Sodium, NF | 12.00 | 14.40 |
| Colloidal Silicon Dioxide, NF | 2.00 | 2.40 |
| Magnesium Stearate, NF | 2.00 | 2.40 |
| Total Tablet Weight | 600.0 | 672.0 |

### Example 3

Large Scale Preparation (300 kg) of a racemic mixture of a piperazine compound formulation

Step (a): Dry Mixture

Pass the racemic mixture of a piperazine compound (100.00 kg), Lactose Monohydrate NF (121.00 kg), Microcrystalline Cellulose NF (51.00 kg), Croscarmellose Sodium NF (6.00 kg) and Colloidal Silicon Dioxide (1.00 kg) through a #10 mesh screen. Add the screened material to a 600 liter Collette mixer. Mix for 6 minutes at low speed, without a chopper.

Step (b): Wet Granulation Mixture

To a stainless steel tank, add Purified Water USP (100.00 kg). While mixing the dry mixture at low speed, pump the purified water into the Collette mixer at a rate of 14 kg/min. After the water has been added, continue to mix the wet granulation mixture at low speed and low chopper for 30 additional seconds. Additional mixing, and/or additional water may be required to achieve the desired consistency. Discharge the wet granulation mixture from the Collette bowl into a suitable transport vessel.

Step (c): Drying Wet Granulation Mixture

Spread the wet granulation evenly, and not to exceed 2 inches in depth, on 2 drying racks lined with 40 lb. Kraft paper. Place the racks in G&G Steam Heated Oven. Dry the wet granulation mixture at 60° C. ±2° C. until a L.O.D. of 1.0-2.1% is reached.

Step (d): Milling the Dried Granulation Mixture

Pass the dried granulation mixture through an auger feed Fitz® mill (Model DAS06), with knives forward, at medium speed, through a 2 Å screen.

Step (e): Mixing a Lubricant

Add the dried granulation mixture from the previous step to a 20-cubic foot V-shell PK blender (Model C266200). Pass Magnesium Stearate NF (1.00 kg) through a 10-mesh screen into a properly prepared container. Add approximately half of the Magnesium Stearate to each side of the PK blender and blend for 5 minutes.

Step (f): Compression into Tablets

Add the blended granulation mixture form the previous step to Kikusui tablet press for compression into capsule-shaped tablets. The compression equipment can be outfitted to make tooling for a 100 mg tablet (0.496×0.218 inches), a 200 mg tablet (0.625×0.275 inches, bisected), 300 mg tablet (0.715×0.315 inches) and a 400 mg tablet (0.750×0.330 inches).

Alternative Step (f): Filling into Capsules

Add the blended granulation mixture form the previous step to H & K 400 machine for filling the appropriate size capsules.

Alternatively, enantiomerically pure compositions, either the R or S enantiomer could replace the racemic mixture as utilized in the preparation of the tablets or capsules.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

1. A pharmaceutical composition comprising a piperazine compound of the formula: 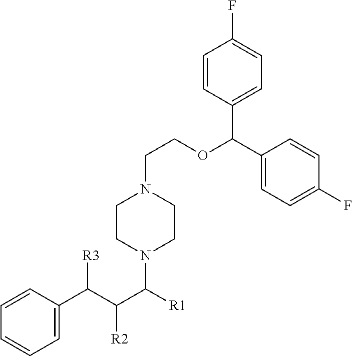
wherein each of R1, R2, and R3 is independently a hydrogen atom or a hydroxyl group, provided that not all of R1, R2 and R3 are the same, and further provided that R1 and R2 are not both a hydroxyl group and that R2 and R3 are not both a hydroxyl group.

2. The composition of claim 1, wherein R1 and R3 are hydrogen atoms and R2 is a hydroxyl group.

3. The composition of claim 1, wherein R1 and R2 are hydrogen atoms and R3 is a hydroxyl group.

4. The composition of claim 1 wherein R1 is a hydroxyl group and R2 and R3 are hydrogen atoms.

5. The pharmaceutical composition of claim 2 wherein the piperazine compound comprises a racemic mixture of the chiral compound.

6. The pharmaceutical composition of claim 2 wherein the piperazine compound comprises an enantiomerically pure amount of the R enantiomer.

7. The pharmaceutical composition of claim 2 wherein the piperazine compound comprises an enantiomerically pure amount of the S enantiomer.

8. The pharmaceutical composition of claim 2 comprising the piperazine compound in an amount of from about 20-50% of the composition by weight; a diluent in an amount of from about 30-60% of the composition by weight; a binder in an amount of from about 15-25% of the composition by weight; a disintegrant in an amount of from about 1-5% of the composition by weight; a flowing agent from about 0.2-0.4% of the composition by weight; and a lubricant from about 0.2-0.4% of the composition by weight.

9. The pharmaceutical composition of claim 8, comprising lactose monohydrate, cross-linked sodium carboxymethylcellulose, colloidal silicon dioxide, and magnesium stearate.

10. The pharmaceutical composition of claim 8, wherein said piperazine compound is present in an amount of from about 30-35% by weight of the composition.

11. The pharmaceutical composition of claim 8, wherein said unit dosage form is a capsule.

12. The pharmaceutical composition of claim 8, wherein said unit dosage form is a tablet.

13. A method for treating cardiac arrhythmias comprising administering a pharmaceutical composition comprising a piperazine compound of the formula: 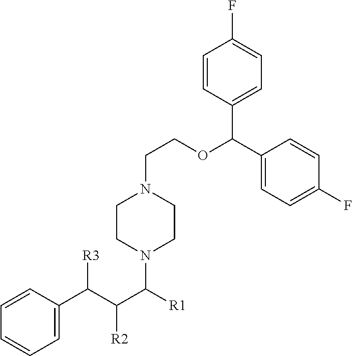
wherein each of R1, R2, and R3 is independently a hydrogen atom or a hydroxyl group, provided that not all of R1, R2 and R3 are the same, and further provided that R1 and R2 are not both a hydroxyl group and that R2 and R3 are not both a hydroxyl group.

14. The method of claim 13 wherein the piperazine compound has the formula wherein R1 and R3 are hydrogen atoms and R2 is a hydroxyl group.

15. The method of claim 13 wherein the piperazine compound has the formula wherein R1 and R2 are hydrogen atoms and R3 is a hydroxyl group.

16. The method of claim 13 wherein the piperazine compound has the formula wherein R1 is a hydroxyl group and R2 and R3 are hydrogen atoms.

17. The composition of claim 14 wherein the piperazine compound comprises a racemic mixture of the chiral compound.

18. The composition of claim 14 wherein the piperazine compound comprises an enantiomerically pure amount of the R enantiomer.

19. The composition of claim 14 wherein the piperazine compound comprises an enantiomerically pure amount of the S enantiomer.
